(19) United States
(12) Patent Application Publication
(10) Pub. No.: US 2013/0324605 A1
(43) Pub. Date: Dec. 5, 2013

(54) USES OF CIMIRACEMATE A AND RELATED COMPOUNDS FOR TREATING INFLAMMATION AND MODULATING IMMUNE RESPONSES

(71) Applicants: Allan Sik Yin Lau, Hong Kong (CN); Cindy Lai Hung Yang, Hong Kong (CN)

(72) Inventors: Allan Sik Yin Lau, Hong Kong (CN); Cindy Lai Hung Yang, Hong Kong (CN)

(73) Assignees: VERSITECH LIMITED, HONG KONG (CN); PURAPHARM COMPANY LIMITED, HONG KONG (CN)

(21) Appl. No.: 13/812,265

(22) PCT Filed: Jul. 29, 2011
(86) PCT No.: PCT/IB11/02582

§ 371 Date: Aug. 23, 2013

Related U.S. Application Data
(60) Provisional application No. 61/369,428, filed on Jul. 30, 2010.

(51) Int. Cl. A61K 31/216

(52) U.S. Cl. CPC A61K 31/216
USPC 514/532

(57) ABSTRACT

The present invention pertains to Cimiracemate A and related compounds that are useful as inhibitors of 5-lipoxygenase (5-1. OX) activity and various proinflammatory mediators such as lipoxins, leukotrienes (e.g., LTA4, LTB4, LTC4, LTD4, and LTE4), prostaglandins, and thromboxanes. The present invention also provides therapeutic methods and compositions for treatment of inflammation and inflammatory conditions, including allergenic reactions, diseases associated with cell proliferation, neoangiogenesis, and cardiovascular diseases.

### CROSS-REFERENCE TO A RELATED APPLICATION

This application claims the benefit of U.S. provisional application Ser. No. 61/369,428, filed Jul. 30, 2010, which is incorporated herein by reference in its entirety, including all figures, tables and sequences.

The Sequence Listing for this application is labeled “As-filed_ST25.txt”, which was created on Jul. 26, 2011, and is 2 KB. The entire contents are incorporated herein by reference in their entirety.

### BACKGROUND OF THE INVENTION

5-lipoxygenase (5-LOX), expressed primarily in leukocytes including monocytes and macrophages, is the rate-limiting enzyme in the biosynthesis of proinflammatory leukotriene lipid mediators. Leukotrienes have been found to be involved in many inflammatory diseases, allergenic reactions, diseases associated with cell proliferation, neoangiogenesis, and cardiovascular diseases²⁻⁹. The biosynthesis of leukotrienes begins with the conversion of arachidonic acid by 5-LOX to the epoxide known as Leukotriene A₄ (LTA₄), which is subsequently converted to other leukotrienes such as Leukotriene B₄ (LTB₄), Leukotriene C₄ (LTC₄), Leukotriene D₄ (LTD₄), and Leukotriene E₄ (LTE₄)⁴².

Leukotrienes, a group of locally acting hormones that actively recruit eosinophils and stimulate the extravasation of plasma, are potent proinflammatory mediators^{20,43}. LTB₄, a non-cysteine-containing dihydroxy leukotriene, is a potent chemoattractant for macrophages, neutrophils, and eosinophils⁴⁴. LTB₄ causes adhesion and chemotactic movement of leukocytes and stimulates aggregation, enzyme release, and generation of superoxide in neutrophils². Cysteinyl leukotrienes (cysLTs), such as LTC₄, LTD₄ and LTE₄, cause mucus secretion, cell migration, inflammatory cell infiltration, increased vascular permeability, tissue edema, ciliary cliren damages, and bronchoconstriction¹⁵. Leukotrienes also increase chloride secretion in colonic mucosa⁴⁵ and cause smooth muscle contractions in a number of tissues including the colon⁴⁶.

It is well documented that blockading the effects of leukotrienes would be beneficial to an array of diseases³³. Recently published studies and case reports have demonstrated that anti-leukotriene agents alleviate symptoms commonly associated with asthma, including exercise-induced asthma, rhinitis, chronic obstructive pulmonary disease, interstitial lung disease, chronic urticaria, atopic dermatitis, allergic fungal diseases, nasal polyposis, and paranasal sinus disease; and non-asthma related diseases, including migraine, respiratory syncytial virus postbronchiolitis, systemic mastocytosis, cystic fibrosis, pancreatitis, vulvovaginal candidiasis, cancer, atherosclerosis, eosinophils cystitis, otitis media, capsular contracture, and eosinophilic gastrointestinal disorders^{2-9, 13-33}.

Inhibition of leukotrienes is also useful to treat diseases, including allergy and inflammation accompanying allergenic diseases of respiratory, gastrointestinal and dermatological systems⁴; tumor and cancer such as urological tumors (e.g. renal cell carcinoma, bladder tumor, prostate cancer⁶ and testicular cancer)³⁴, pancreatic cancer⁵; adipose tissue inflammation; cerebrovascular and cardiovascular diseases such as neoangiogenesis, myocardial infarction⁸, acute myocardial infarction⁴⁷, stroke⁸, atherosclerosis⁹, thrombosis, coronary angioplasty²⁰, aortic aneurysms²⁰, vascular inflammation²⁰, intimal hyperplasia²⁰, hyperlipidemia-dependent aortic aneurysm⁸; cystic fibrosis lung diseases²⁷; sleep-disorder breathing²⁴; obstructive sleep apnea (OSA)²⁴, chronic inflammatory bowel diseases⁴⁸; Crohn's disease⁴⁹; allergic rhinitis³³; fractured bone¹⁶; arthritis; and chronic obstructive pulmonary disease (COPD)¹³⁻¹⁵.

The capacity of leukotrienes to act as potent proinflammatory mediators drives the development of anti-leukotriene agents, which are recognized as high potential therapeutics for a variety of diseases. Anti-leukotrienes are classified into two major categories: leukotriene receptor antagonists and synthesis inhibitors³³. Although leukotriene receptor antagonist drugs such as montelukast, zafirlukast and pranlukast represent important therapeutic advances, particularly in the treatment of asthma, clinical studies have revealed that these leukotriene receptor antagonists cause side effects such as gastrointestinal disturbances, hypersensitivity reactions, sleep disorders, increased bleeding tendency, vasculitic rash, worsening of pulmonary symptoms, and cardiac complications. Use of montelukast is also reported as associated with higher incidence of Churg-Strauss syndrome and increased risk of neuropsychiatric disturbance, including of insomnia, agitation, aggression, anxiousness, dream abnormalities, hallucinations, depression, and suicidal thoughts. Therefore, there is a need for developing alternative anti-leukotriene agents with improved efficacy and safety.

Traditional Chinese medicine has been practiced by the Chinese people for 2-3 millennia. It deals with pathology, and diagnosis, treatment and prevention of diseases. Chinese medicinal materials have been recorded in various pharmacopoeia. One of the classical references for medicinal herbs is Ben Cao Gang Mu written by Li, Shizhen in the late 14^{th} Century. The book contains about 2,500 items of herbs and other products including animals and minerals.

Various species of Cimicifuga have been used as therapeutics for inflammatory conditions in Chinese, Korean, and Japanese medicine. Cimicifuga racemosa (also known as black cohosh) and its counterparts Cimicifuga heracleifolia, Cimicifuga foetida and Cimicifuga dahurica have been used as traditional medicinal herbs to treat fever, pain and inflammation in Asian countries including China, Japan and Korea. Cimicifuga species also have a long and diverse history of medicinal use in the United States and Canada. As a result, the toxicity of the herbs as well as their chemical constituents in human uses has been well tested for centuries.

Cimiracemate A, an ester formed between isoferulic acid and 3-(30,40-dihydroxylphenyl)-2-keto-propanol, is a naturally-occurring compound that can be isolated from many Cimicifuga species including Cimicifuga racemosa. The present inventors have identified that Cimiracemate A suppresses LPS-induced TNF-α in human macrophages and inhibits LPS-induced MAP kinase activities¹². Cimiracemate A may have additional health benefits, including acting as reactive oxygen species scavengers³⁵. Cimiracemate A and its related compounds, however, have not previously been reported to play any role in inhibition of 5-lipoxygenase (5-LOX) activity or leukotriene biosynthesis.

### BRIEF SUMMARY

The present invention pertains to novel uses of compounds of Formula I and compositions comprising these compounds as 5-lipoxygenase (5-LOX) inhibitors and anti-leukotriene agents. The compounds have the following structure:

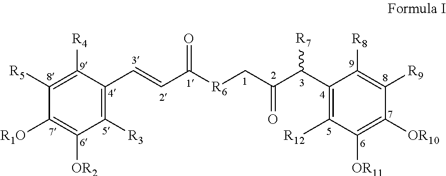

wherein
R₁ is alkyl;
R₂ is H or alkyl;
R₃, R₄, and R₅ are independently —H, acyl, halo, haloalkyl, amino, alkylamino, hydroxyl, alkyl, hydroxylalkyl, or —COOH;
R₆ is —O or —NH;

R₇ is —H, alkyl, alkoxy, hydroxylalkyl, hydroxyl, or halo;
R₈, R₉, and R₁₂ are independently —H, acyl, halo, amino, alkylamino, hydroxyl, alkyl, hydroxylalkyl, or —COOH;
R₁₀ is H or alkyl; and
R₁₁ is H or alkyl.

The present compounds and compositions are useful to treat inflammatory diseases, allergenic reactions, diseases associated with cell proliferation, neoangiogenesis, and cardiovascular diseases. In certain embodiments, due to the modulation effects of these compounds on 5-lipoxygenase and TNF-α, they have immunomodulatory activity that is not specifically associated with inflammation.

One embodiment of the present invention pertains to uses of Cimiracemate A. Further embodiment is uses of Cimiracemate A isolated from herbs such as Cimicifuga species. Advantageously, Cimiracemate A potently inhibits 5-lipoxygenase activity and blocks biosynthesis of lipoxins, leukotrienes (e.g., LTA₄, LTB₄, LTC₄, LTD₄, and LTE₄), prostaglandins, and thromboxanes.

The present invention is also directed to pharmaceutical compositions comprising a pharmaceutically acceptable carrier and a compound of Formula I. In a preferred embodiment, the composition contains the compound of Formula I as the active ingredient.

In certain embodiments, the compounds and pharmaceutical compositions of the present invention are useful to treat or ameliorate diseases, such as for example, asthma, exercise-induced asthma, rhinitis, chronic obstructive pulmonary disease, interstitial lung disease, chronic urticaria, atopic dermatitis, allergic fungal diseases, nasal polyposis, paranasal sinus disease, migraine, respiratory syncytial virus postbronchiolitis, systemic mastocytosis, cystic fibrosis, pancreatitis, vulvovaginal candidiasis, cancer, atherosclerosis, eosinophils cystitis, otitis media, capsular contracture, and eosinophilic gastrointestinal disorders.

### BRIEF DESCRIPTION OF THE DRAWINGS

FIGS. 1A-B show inhibitory effects of Cimiracemate A on 5-lipoxygenase (5-LOX) and production of Leukotriene B₄ (LTB₄).

FIG. 2 shows an extraction scheme of Cimiracemate A (B22EES1-8-3) from Cimicifuga racemosa. Cimicifuga racemosa (1.8 kg) was milled and extracted with 500 mL milli-Q water for 1 hr with continuous sonication. The collected supernatant was then partitioned with ethyl acetate (EtOAc) (1:1). The resulting dried EtOAc extract was reconstituted and then sequentially partitioned with hexane (n-C₆H₁₄), EtOAc and butanol (n-BuOH). Using bioassay guided fractionation scheme, the fractions showing inhibitory effects on LPS-induced TNF-α production were subjected to silica gel 60A (35-75 μm) chromatography and reversed-phase high-performance liquid chromatography using gradient elution until a single compound with anti-inflammatory activity was obtained.

FIGS. 3A-3B show HPLC chromatogram and UV absorbance of B22EES1-8-3. The compound was purified by reversed-phase HPLC using gradient elution from 25% to 90% of acetonitrile at a flow rate of 1 mL min⁻¹. (A) A single peak was detected using Photo-diode Array detector at 254, 210 and 280 nm. B22EES1-8-3 was eluted at approximate 9.4 min. (B) The UV absorbance of B22EES1-8-3 maximized at 290 and 325 nm which revealed that it had a conjugated aromatic system.

FIG. 4 shows the ¹H (upper panel) and ¹³C NMR (lower panel) spectra of B22EES1-8-3. The structure of B22EES1-8-3 was elucidated by a Bruker 500 MHz DRX NMR spectrometer, operating at 500 MHz for ¹H and at 125.765 MHz for ¹³C NMR, using methanol-d as the solvent.

FIGS. 5A-5B show a bioassay guided fractionation of Cimicifuga racemosa. Primary blood macrophages (PBMac) were treated with different C. racemosa fractions at 100 μg/mL for 24 hr prior to the addition of 20 ng/mL LPS for 3 hr. RT-PCR (A) and quantitative RT-PCR (B) assays of TNF-α and GAPDH were performed afterwards. The results shown are representative of at least three independent experiments, with cells obtained from different donors. * P<0.05, compared with the corresponding control.

FIGS. 6A-6B show inhibition of LPS-induced TNF-α production by B22EES1-8-3 and dexamethasone. PBMac were incubated with (A) 140 μM B22EES1-8-3 or (B) 1.3 or 5.1 μM dexamethasone (Dex) for 24 hr prior to the addition of 1 ng/mL and 10 ng/mL LPS for another 24 hr. The culture supernatants were collected and assayed for TNF-α by ELISA. The results shown were the mean values±standard derivation (S.D.) of 6 independent experiments, with cells obtained from different donors. * P<0.05, compared with the corresponding control.

FIGS. 7A-7C show the effects of Cim A (B22EES1-8-3) on LPS-induced phosphorylation (phospho-) of ERK1/2 and p38 MAP kinases, and nuclear translocation of NF-KB p65. PBMac were incubated with B22EES1-8-3 (140 μM) for 24 h prior to the addition of 10 ng/mL LPS for an additional 15 min. Cytoplasmic (A, B) and nuclear (C) proteins were harvested for Western Blotting: (A) Cytoplasmic proteins: phospho-ERK1/2 and total ERK1/2. (B) Cytoplasmic proteins: phospho-p38 and total p38 kinase. (C) Nuclear proteins: upper panel, NF-κB p65 and lamin B; lower panel, the intensity of corresponding lanes in the gel photograph of NF-κB p65 was shown. The results shown are representative of at least three independent experiments, with cells obtained from different donors. * P<0.05, compared with the corresponding control.

FIGS. 8A-8B show the HPLC chromatograms of CF22EES1-8 (A) and CH22EES1-8 (B). Herbs C. foetida and C. heracleifolia were extracted following the extraction procedure of C. racemosa. Their extracts (CF22EES1-8 and CH22EES1-8) were injected into the HPLC using the same condition as that of B22EES1-8-3 and the chromatograms were recorded. The chromatograms showed the presence of a compound (with *) with retention time at approximate 9.4 minutes.

FIGS. 9A-9C show the UPLC chromatograms and HRESI-MS spectra of (A) B22EES1-8-3, (B) CF22EES1-8, and (C) CH22EES1-8. Herbs C. foetida and C. heracleifolia were extracted following the extraction procedure of C. racemosa. Their fractions (CF22EES1-8 and CH22EES1-8) were injected into an UPLC-coupled high-resolution ESI-TOF-MS using the same condition as that of B22EES1-8-3. The chromatograms showed the presence of a compound (with *) with retention time at approximately 6 min and with an ion peak at 357 m/z.

### BRIEF DESCRIPTION OF THE SEQUENCES

SEQ ID NO:1 is a primer useful according to the present invention.

SEQ ID NO:2 is a primer useful according to the present invention.

SEQ ID NO:3 is a primer useful according to the present invention.

SEQ ID NO:4 is a primer useful according to the present invention.

### DETAILED DESCRIPTION

The present invention pertains to novel uses, as 5-lipoxygenase (5-LOX) inhibitors and anti-leukotriene agents, of compounds of Formula I and compositions comprising these compounds. The compounds and compositions of the present invention are useful to treat or ameliorate inflammatory diseases, allergenic reactions, diseases associated with cell proliferation, neoangiogenesis, and cardiovascular diseases. Specifically exemplified herein is the therapeutic use of Cimiracemate A (Cim A).

### Compounds

The present invention provides therapeutic compounds of Formula I, which are potent 5-lipoxygenase (5-LOX) inhibitors and anti-leukotriene agents. The compounds have the following structure:

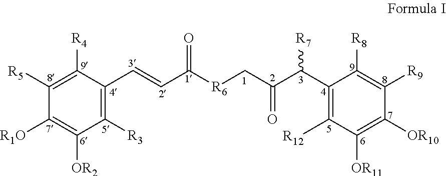

### Wherein

R₁ is alkyl;
R₂ is H or alkyl;
R₃, R₄, and R₅ are independently —H, acyl, halo, haloalkyl, amino, alkylamino, hydroxyl, alkyl, hydroxylalkyl, or —COOH;
R₆ is —O or —NH;
R₇ is —H, alkyl, alkoxy, hydroxylalkyl, hydroxyl, or halo;
R₈, R₉, and R₁₂ are independently —H, acyl, halo, amino, alkylamino, hydroxyl, alkyl, hydroxylalkyl, or —COOH;
R₁₀ is H or alkyl; and
R₁₁ is H or alkyl.
   “Alkyl” means linear saturated monovalent radicals of one to eight carbon atoms or a branched saturated monovalent of three to eight carbon atoms. It may include hydrocarbon radicals of one to four or one to three carbon atoms, which may be linear. Examples include methyl, ethyl, propyl, 2-propyl, n-butyl, iso-butyl, tert-butyl, pentyl, and the like.
   “Acyl” means a radical —C(O)R where R is hydrogen, alkyl or cycloalkyl, or heterocycloalkyl. Examples include formyl, acetyl, ethylcarbonyl, and the like.
   “Halo” means fluoro, chloro, bromo, or iodo, such as bromo and chloro.
   “Haloalkyl” means alkyl substituted with one or more same or different halo atoms, e.g. —CH₂Cl, —CH₂Br, —CF₃, —CH₂CH₂Cl, —CH₂CCl₃, and the like.
   An “amino” is intended to mean the radical —NH₂.
   “Alkylamino” means a radical —NHR or —NR₂ where each R is independently an alkyl group. Examples include methylamino, (1-methylethyl)amino, methylamino, dimethylamino, methylethylamino, di(1-methylethyl)amino, and the like.
   A “hydroxy” is intended to mean the radical —OH.
   Hydroxyalkyl” means an alkyl radical as defined herein, substituted with one or more, preferably one, two or three hydroxy groups. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxy-propyl, preferably 2-hydroxyethyl, 2,3-dihydroxypropyl and 1-(hydroxymethyl) 2-hydroxyethyl.
   An “alkoxy” is intended to mean the radical —ORₐ, where Rₐ is an alkyl group. Exemplary alkoxy groups include methoxy, ethoxy, propoxy, and the like.

The present invention further pertains to isolated enantiomeric compounds. The isolated enantiomeric forms of the compounds of the invention are substantially free from one another (i.e., in enantiomeric excess). In other words, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. In one embodiment of the invention, the isolated enantiomeric compounds are in at least about 80% enantiomeric excess. In a preferred embodiment, the compounds are in at least about 90% enantiomeric excess. In a more preferred embodiment, the compounds are in at least about 95% enantiomeric excess. In an even more preferred embodiment, the compounds are in at least about 97.5% enantiomeric excess. In a most preferred embodiment, the compounds are in at least about 99% enantiomeric excess. In this context, the applicants incorporate herein by reference, in its entirety, International Application No.: PCT/IB2009/055970.

In an embodiment, the present invention pertains to uses of isolated compounds. The compounds of Formula I can be isolated from a medicinal plant such as Cimicifuga species, including Cimicifuga racemosa, Cimicifuga heracleifolia, and Cimicifuga foetida.

As used herein, “isolated” refers to compounds that have been removed from any environment in which they may exist in nature. For example, isolated Cim A would not refer to the Cim A compound as it exists in Cimicifuga species such as C. racemosa. In preferred embodiments, the compounds of the present invention are at least 75% pure, preferably at least 90% pure, more preferably are more than 95% pure, and most preferably are more than 99% pure (substantially pure).

In one embodiment, the present invention pertains to therapeutic uses of compounds of Formula I, wherein R₂ is H, R₃ is H, and R₄ is H. In another embodiment, the present invention pertains to therapeutic uses of compounds of Formula I, wherein R₁ is a methyl group.

In a specific embodiment, the present invention pertains to therapeutic uses of Cimiracemate A (Cim A), having the following structure:

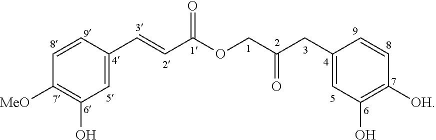 [SMILES: COc1ccc(/C=C/C(=O)OCC(=O)Cc2ccc(O)c(O)c2)cc1O]

Surprisingly, it has now been discovered that Cim A and its related compounds potently inhibit 5-LOX activity and block the biosynthesis of leukotrienes such as LTB₄. It has been reported that 5-LOX inhibition blocks biosynthesis and activity of a wide range of proinflammatory mediators, including lipoxins, leukotrienes, prostaglandins, and thromboxanes². Particularly, it is well documented that inhibition of 5-LOX blocks biosynthesis of leukotrienes. Therefore, the present compounds are useful for preventing, reversing or alleviating pathological conditions induced by leukotrienes.

In addition, Cim A and its related compounds can inhibit aldose reductase, cyclooxygenase, HIV integrase, adrenergic (beta1), and phospholipase A2 activity. Cim A can also be used to reduce levels of cAMP and CGMP.

In addition, the present inventors have discovered that Cim A inhibits TNF-α induction and abrogates MAP kinase and NF-KB activation¹². TNF-α, MAP kinase and NF-KB are mediators that play a key role in production of cytokine and regulation of a range of immune responses. Therefore, Cim A can also be used to regulate the downstream effectors of TNF-α.

The present inventors have also discovered that the effects of Cim A on the regulation of cytokines occur via its activity in the modulation of signaling kinase and transcription factor activities. Cim A suppresses mitogen induced inflammatory response, which makes this molecule useful for treatment of a variety of clinical conditions. Since overproduction of TNF-α is toxic and can result in severe complications, limiting the overwhelming inflammatory response can be beneficial to patients in clinical management.

Cim A can be isolated from Cimicifuga species, such as Cimicifuga racemosa, Cimicifuga heracleifolia, and Cimicifuga foetida using unique isolation and bioassay-guided procedures developed by the present inventors (Examples 3-4)¹².

Treatment of Inflammatory Diseases and/or Immune Disorders

The present invention provides methods for treatment or amelioration of inflammatory diseases and/or immune disorders, particularly diseases or disorders associated with 5-Lipoxygenase activity and/or leukotriene over-production. In one embodiment, the present invention provides methods for treatment or amelioration of inflammatory diseases and/or immune disorders that are not disclosed as diseases that are to be treated in accordance with the teachings in International Application No.: PCT/IB2009/055970.

In certain embodiments, the compounds and compositions of the present invention are useful to treat or ameliorate conditions, including but not limited to, inflammation, allergenic reactions, diseases associated with cell proliferation, neoangiogenesis, and cardiovascular diseases. The method comprises administering, to a subject in need of such treatment, an effective amount of the compounds and compositions of the present invention.

The compounds and compositions of the present invention are particularly useful to treat or ameliorate diseases in which inhibition of synthesis and activity of lipoxins, leukotrienes (e.g., LTA₄, LTB₄, LTC₄, LTD₄, and LTE₄), prostaglandins, and thromboxanes would be beneficial.

The term “effective amount,” as used herein, refers to an amount that is capable of treating or ameliorating a disease or condition or otherwise capable of producing an intended therapeutic effect. In certain embodiments, the effective amount enables a 5%, 10%, 20%, 30%, 50%, 60%, 70%, 80%, 90%, 95%, 99% and 100% reduction in 5-LOX activity. Additionally or alternatively, the effective amount enables a 5%, 10%, 20%, 30%, 50%, 60%, 70%, 80%, 90%, 95%, 99% and 100% reduction in the levels and/or biosynthesis of lipoxins, leukotrienes (e.g., LTA₄, LTB₄, LTC₄, LTD₄, and LTE₄), prostaglandins, and/or thromboxanes.

The term “subject,” as used herein, describes an organism, including mammals such as primates, to which treatment with the compositions according to the present invention can be provided. Mammalian species that can benefit from the disclosed methods of treatment include, but are not limited to, apes, chimpanzees, orangutans, humans, monkeys; and domesticated animals such as dogs, cats, horses, cattle, pigs, sheep, goats, chickens, mice, rats, guinea pigs, and hamsters.

The compounds and pharmaceutical compositions of the present invention can be used in the treatment or amelioration of inflammatory symptoms in any disease, condition or disorder where immune and/or inflammation suppression is beneficial. Inflammatory diseases, conditions or disorders in which the compounds and compositions of the present invention can be used to inhibit unwanted immune reactions and inflammation include, but are not limited to, arthritis, including but not limited to rheumatoid arthritis, and other diseases, conditions or disorders of the joints or musculoskeletal system in which immune and/or inflammation suppression is beneficial.

The compounds and pharmaceutical compositions of the present invention are useful to treat or ameliorate asthma and asthma-associated diseases, such as for example, exercise-induced asthma, rhinitis, chronic obstructive pulmonary disease, interstitial lung disease, chronic urticaria, atopic dermatitis, allergic fungal diseases, nasal polyposis, and paranasal sinus disease.

In addition, the compounds and pharmaceutical compositions of the present invention are useful to treat or ameliorate non-asthma-related diseases, including migraine, respiratory syncytial virus postbronchiolitis, systemic mastocytosis, cystic fibrosis, pancreatitis, vulvovaginal candidiasis, cancer, atherosclerosis, eosinophils cystitis, otitis media, capsular contracture, and eosinophilic gastrointestinal disorders.

In addition, the compounds and pharmaceutical compositions of the present invention are useful to treat or ameliorate diseases, including allergy and inflammation accompanying allergenic diseases of respiratory, gastrointestinal and dermatological systems; adipose tissue inflammation; chronic inflammatory bowel diseases; Crohn's disease; allergic rhinitis; fractured bone; arthritis; and tumor and cancer such as urological tumors (e.g. renal cell carcinoma, bladder tumor, prostate cancer and testicular cancer), neoangiogenesis, and pancreatic cancer.

In addition, the compounds and pharmaceutical compositions of the present invention are useful to treat or ameliorate diseases, including cerebrovascular and cardiovascular diseases such as myocardial infarction, acute myocardial infarction, stroke, atherosclerosis, thrombosis, coronary angioplasty, aortic aneurysms, vascular inflammation, intimal hyperplasia, hyperlipidemia-dependent aortic aneurysm; cystic fibrosis lung diseases; sleep-disorder breathing, obstructive sleep apnea (OSA); and chronic obstructive pulmonary disease (COPD).

In addition, the compounds and pharmaceutical compositions of the present invention are useful to treat or ameliorate diseases, including pulmonary disorders including diseases such as asthma, chronic bronchitis, and related obstructive airway diseases; allergies and allergic reactions such as allergic rhinitis, contact dermatitis, allergic conjunctivitis, and the like; inflammation such as arthritis or inflammatory bowel disease; pain; skin disorders such as psoriasis, atopic eczema, and the like; cardiovascular disorders such as angina, myocardial ischemia, hypertension, platelet aggregation and the like; renal insufficiency arising from ischemia induced by immunological or chemical (cyclosporin) etiology; migraine or cluster headache; ocular conditions such as uveitis; hepatitis resulting from chemical, immunological or infectious stimuli; trauma or shock states such as burn injuries, endotoxemia and the like; allograft rejection; chronic lung diseases such as cystic fibrosis, bronchitis and other small and large-airway diseases; and cholecystitis.

Moreover, the compounds and compositions are also useful to treat or ameliorate inflammation associated with atherosclerosis; arteriosclerosis; atherosclerotic heart disease; reperfusion injury; cardiac arrest; myocardial infarction; vascular inflammatory disorders including cerebrovascular disease (stroke); respiratory distress syndrome and other cardiopulmonary diseases, conditions or disorders where immune and/or inflammation suppression would be beneficial.

In addition, the compounds and compositions are also useful to treat or ameliorate inflammation associated with peptic ulcer; ulcerative colitis, Crohn's Disease, irritable bowel syndrome, other inflammatory bowel conditions, and other diseases, conditions or disorders of the gastrointestinal tract where immune inflammation suppression would be beneficial; hepatic fibrosis; liver cirrhosis and other hepatic diseases, conditions or disorders where immune and/or inflammation suppression would be beneficial; thyroiditis and other glandular diseases, conditions or disorders where immune and/or inflammation suppression would be beneficial; glomerulonephritis and other renal and urologic diseases, conditions or disorders where immune and/or inflammation suppression would be beneficial.

In addition, the compounds and compositions are also useful to treat or ameliorate inflammation associated with post-traumatic inflammation; septic shock; infectious diseases where immune and/or inflammation suppression would be beneficial; inflammatory complications and side effects of surgery where immune and/or inflammation suppression would be beneficial; bone marrow transplantation and other transplantation complications and/or side effects where immune and/or inflammation suppression would be beneficial; inflammatory and/or immune complications and side effects of gene therapy, e.g., due to infection with a viral carrier; and inflammation associated with acquired immune deficiency syndrome (AIDS).

Further, the compounds and compositions are also useful to inhibit macrophage or T cell associated aspects of an immune response that are not associated with inflammation. The compounds and compositions are able to inhibit macrophage or T cell activities including, but not limited to, macrophage antigen-presenting activity, macrophage cytokine production, T cell cytokine production, T cell adhesion activity, T cell proliferation, etc. Thus, the peptides, peptide derivatives and compositions are useful to suppress or inhibit a humoral and/or cellular immune response.

The compounds and compositions are also useful to treat or ameliorate monocyte and leukocyte proliferative diseases, e.g., leukemia, by reducing the amount of monocytes and lymphocytes.

The compounds and pharmaceutical compositions of the invention are further useful for the prevention and/or treatment of graft rejection in cases of transplantation of natural or artificial cells, tissue and organs, such as cornea, bone marrow, organs, lenses, pacemakers, natural and artificial skin tissue, and the like.

The compounds and compositions are also useful to treat or ameliorate inflammation associated with hypersensitivity; allergic reactions; asthma; systemic lupus erythematosus; collagen diseases and other autoimmune diseases, conditions or disorders in which immune and/or inflammation suppression is beneficial.

The compounds and compositions are also useful to treat or ameliorate inflammation associated with otitis and other otorhinolaryngological diseases, conditions or disorders where immune and/or inflammation suppression would be beneficial; dermatitis and other dermal diseases, conditions or disorders where immune and/or inflammation suppression would be beneficial; periodontal diseases and other dental diseases, conditions or disorders where immune and/or inflammation suppression would be beneficial.

In addition, the compounds and compositions are also useful to treat or ameliorate inflammation associated with posterior uveitis; intermediate uveitis; anterior uveitis; conjunctivitis; chorioretinitis; uveoretinitis; optic neuritis; intraocular inflammation, such as retinitis and cystoid macular edema; sympathetic ophthalmia; scleritis; retinitis pigmentosa; immune and inflammatory components of degenerative fondus disease; inflammatory components of ocular trauma; ocular inflammation caused by infection; proliferative vitreoretinopathies; acute ischemic optic neuropathy; excessive scarring, for example, following glaucoma filtration operation; immune and/or inflammation reaction against ocular implants and other immune and inflammatory-related ophthalmic diseases, conditions or disorders where immune and/or inflammation suppression would be beneficial.

Moreover, the compounds and compositions are also useful to treat or ameliorate inflammation associated with autoimmune diseases and conditions or disorders where, both in the central nervous system (CNS) and in any other organ, immune and/or inflammation suppression would be beneficial; Parkinson's disease; complications and/or side effects from treatment of Parkinson's disease; AIDS-related dementia complex (HIV-related encephalopathy); Devic's disease; Sydenham chorea; Alzheimer's disease and other degenerative diseases, conditions or disorders of the central nervous system where immune and/or inflammation suppression would be beneficial; inflammatory components of strokes; post-polio syndrome; immune and inflammatory components of psychiatric disorders; myelitis; encephalitis; subacute sclerosing panencephalitis; encephalomyelitis; acute neuropathy; subacute neuropathy; chronic neuropathy; Guillain-Barre syndrome; Sydenham chorea; myasthenia gravis; pseudotumor cerebri; Down's Syndrome; Huntington's disease; amyotrophic lateral sclerosis; inflammatory components of central nervous system (CNS) compression or CNS trauma or cerebrovascular accidents (stroke) or infections or hypoxia-ischemia of the CNS; inflammatory components of muscular atrophies and dystrophies; and immune and inflammatory related diseases, conditions or disorders of the central and peripheral nervous systems where immune and/or inflammation suppression would be beneficial.

In yet another embodiment, the compounds and compositions of the invention are useful to restore immune privilege at an immune privileged site which has lost its immune privilege such as brain, eye and testis.

In a further embodiment, the present method comprises: determining the level of one or more biomarker (such as 5-LOX, leukotrienes (e.g., LTA₄, LTB₄, LTC₄, LTD₄, and LTE₄), prostaglandins, lipoxins, thromboxane, TNF-α, LPS, NF-KB, cAMP, CGMP, and MAP kinase) in a subject, and prescribing the compounds and compositions of the invention if reduction of the level of said one or more biomarkers would produce beneficial therapeutic effects.

### Therapeutic Compositions and Formulations

The present invention also provides for therapeutic or pharmaceutical compositions comprising a compound of the invention in a form that can be combined with a pharmaceutically acceptable carrier. In this context, the compound may be, for example, isolated or substantially pure. The term “carrier” refers to a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions. Particularly preferred pharmaceutical carriers for treatment of or amelioration of inflammation in the central nervous system are carriers that can penetrate the blood/brain barrier. As used herein carriers do not include the natural plant material as it exists in nature.

The present invention also pertains to uses of prodrugs and metabolites of the compounds. The term “prodrug,” as used herein, refers to a metabolic precursor of a compound of the present invention or pharmaceutically acceptable form thereof. In general, a prodrug comprises a functional derivative of a compound, which may be inactive when administered to a subject, but is readily convertible in vivo into an active metabolite compound. Conventional procedures for the selection and preparation of suitable prodrug derivatives are described, for example, in “Design of Prodrugs”, ed. H. Bundgaard, Elsevier, 1985. Preferably, a prodrug of the present invention enhances desirable qualities of the compound of the present invention including, but not limited to, solubility, bioavailability, and stability. Hence, the compounds employed in the present methods may, if desired, be delivered in a prodrug form. Prodrugs of the compounds employed in the present invention may be prepared by modifying functional groups present in the compound such that the modifications are cleaved, either in routine manipulation or in vivo, to the parent compound.

The term “metabolite,” refers to a pharmacologically active product, including for example, an active intermediate or an ultimate product, produced through in vivo metabolism of a compound of the present invention in a subject. A metabolite may result, for example, from the anabolic and/or catabolic processes of the administered compound in a subject, including but not limited to, the oxidation, reduction, hydrolysis, amidation, deamidation, esterification, deesterification, enzymatic cleavage, and the like.

Metabolites are typically identified by preparing a radiolabelled (e.g., ¹⁴C or .³H) isotope of a compound of the present invention, administering it parenterally in a detectable dose (e.g., greater than about 0.5 mg/kg) to an animal such as rat, mouse, guinea pig, monkey, or to a human, allowing sufficient time for metabolism to occur (typically about 30 seconds to about 30 hours), and isolating its conversion products from the urine, blood or other biological samples. These products are easily isolated since they are labeled (others are isolated by the use of antibodies capable of binding epitopes surviving in the metabolite). The structure of metabolites can be determined in conventional fashion, e.g., by MS, LC/MS or NMR analysis. In general, analysis of metabolites is performed according to techniques well known to those skilled in the art of drug metabolism studies.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The therapeutic composition, if desired, can also contain minor amounts of wetting or emulsifying agents, or pH buffering agents. These compositions can take the form of solutions, suspensions, emulsion, tablets, capsules, powders, sustained-release formulations and the like. The composition can be formulated with traditional binders and carriers such as triglycerides. Examples of suitable pharmaceutical carriers are described in “Remington's Pharmaceutical Sciences” by E. W. Martin. Such compositions contain a therapeutically effective amount of the therapeutic composition, together with a suitable amount of carrier so as to provide the form for proper administration to the patient. The formulation should suit the mode of administration.

In one embodiment, the composition is formulated in accordance with routine procedures as a pharmaceutical composition adapted for local injection administration to human beings. Typically, compositions for local injection administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The therapeutic or pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The present invention also provides for the modification of the compound such that it is more stable once administered to a subject, i.e., once administered it has a longer time period of effectiveness as compared to the unmodified compound. Such modifications are well known to those of skill in the art, e.g., microencapsulation, etc. The amount of the therapeutic or pharmaceutical composition of the invention which is effective in the treatment of a particular disease, condition or disorder will depend on the nature of the disease, condition or disorder and can be determined by standard clinical techniques. In general, the dosage ranges from about 0.001 mg/kg to about 500 mg/kg. In addition, in vitro assays may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems. For example, in order to obtain an effective mg/kg dose for humans based on data generated from rat studies, the effective mg/kg dosage in rats is divided by six.

For instance, suitable unit dosages may be between about 0.01 to about 500 mg, about 0.01 to about 300 mg, about 0.01 to about 200 mg, about 0.01 to about 100 mg, about 0.01 to about 50 mg, about 0.01 to about 30 mg, about 0.01 to about 20 mg, about 0.01 to about 10 mg, about 0.01 to about 5 mg, about 0.01 to about 3 mg, about, 0.01 to about 1 mg, or about 0.01 to about 0.5 mg. Such a unit dose may be administered more than once a day, e.g. two or three times a day.

The invention also provides a pharmaceutical pack or kit comprising one or more containers filled with one or more of the ingredients, e.g., compound, carrier, of the pharmaceutical compositions of the invention.

The compounds of the present invention can also be formulated consistent with traditional Chinese medicine practices. The composition and dosage of the formulation that are effective in the treatment of a particular disease, condition or disorder will depend on the nature of the disease, condition or disorder by standard clinical techniques.

The traditional Chinese medicine in prescription amounts can be readily made into any form of drug, suitable for administering to humans or animals. Suitable forms include, for example, tinctures, decoctions, and dry extracts. These can be taken orally, applied through venous injection or mucous membranes. The active ingredient can also be formulated into capsules, granules, powder, pallets, pastille, suppositories, oral solutions, pasteurized gastroenteric suspension injections, small or large amounts of injection, frozen powder injections, pasteurized powder injections and the like. All of the above-mentioned methods are known to people skilled in the art, described in books and commonly used by practitioners of herbal medicine.

In preferred embodiments, the compounds of the present invention are prepared as discrete units such as granules (e.g., wet granules, dry granules); capsules, cachets or tablets, each containing a predetermined amount of the active ingredient.

The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80%, or about 30% to about 70% active ingredient (w/w).

A tincture is prepared by suspending herbs in a solution of alcohol, such as, for example, wine or liquor. After a period of suspension, the liquid (the alcohol solution) may be administered for example, two or three times a day, one teaspoon each time.

A decoction is a common form of herbal preparation. It is traditionally prepared in a clay pot, but can also be prepared in glass, enamel or stainless steel containers. The formulation can be soaked for a period of time in water and then brought to a boil and simmered until the amount of water is reduced by, for example, half.

An extract is a concentrated preparation of the essential constituents of a medicinal herb. Typically, the essential constituents are extracted from the herbs by suspending the herbs in an appropriate choice of solvent, typically, water, ethanol/water mixture, methanol, butanol, iso-butanol, acetone, hexane, petroleum ether or other organic solvents. The extracting process may be further facilitated by means of maceration, percolation, repercolation, counter-current extraction, turbo-extraction, or by carbon-dioxide hypercritical (temperature/pressure) extraction. After filtration to rid of herb debris, the extracting solution may be further evaporated and thus concentrated to yield a soft extract (extractum spissum) and/or eventually a dried extract, extracum siccum, by means of spray drying, vacuum oven drying, fluid-bed drying or freeze-drying. The soft extract or dried extract may be further dissolved in a suitable liquid to a desired concentration for administering or processed into a form such as pills, capsules, injections, etc.

### Materials and Methods

### Plant Material

Cimicifuga racemosa was purchased from the Glenbrook Farms Herbs and Such, Campbellsville, Ky. Cimicifuga heracleifolia, Cimicifuga foetida and Cimicifuga dahurica were purchased in herbal markets and subsequently authenticated by Purapharm with respect to their identification.

### Extraction and Isolation of the Bioactive Molecules

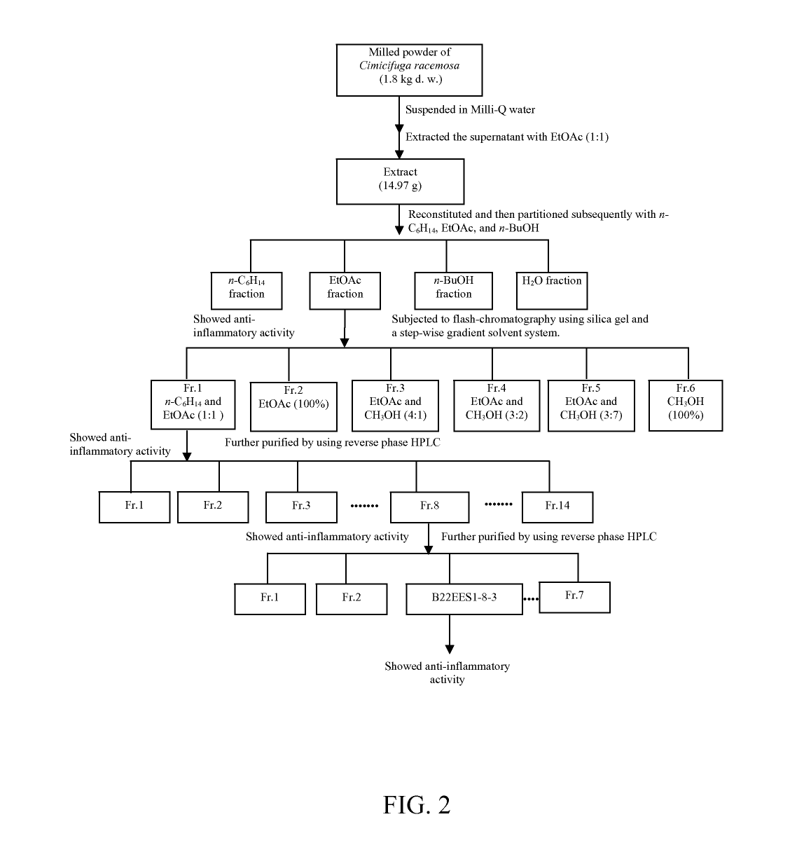

The procedures for plant extraction are shown in FIG. 2. Briefly, Cimicifuga racemosa (1.8 kg) was milled, homogenized and then suspended in (1:5) milli-Q water for 1 hr with continuous sonication. The supernatant was filtered through an analytical filter paper and then partitioned three times with ethyl acetate (EtOAc) (1:1). The resulting EtOAc extract was concentrated to dryness in vacuo (35° C.) to yield 14.97 g of a dark brown residue. The residue was reconstituted in methanol (MeOH) and then fractionated by partitioning with hexane (n-C₆H₁₄). The MeOH fraction was concentrated and reconstituted in H₂O and then partitioned sequentially with EtOAc and butanol (n-BuOH). Four fractions, namely n-C₆H₁₄, EtOAc, n-BuOH, and H₂O were obtained.

The fraction that showed inhibitory effects on LPS-induced TNF-α production was subjected to additional silica gel 60A (35-75 μm) chromatography using n-C₆H₁₄, EtOAc, and MeOH to yield six fractions. The active fractions were further purified by reversed-phase high-performance liquid chromatography (HPLC) (Lichrospher 100 RP C18 EC 5μ, 250×4.6 mm ID) using a gradient elution from 25% acetonitrile (CH₃CN) to 90% CH₃CN at a flow rate of 1 mL min⁻¹.

Peak detection was achieved using an Agilent 1200 series of fast scanning Photo-diode Array detector set at 254, 210 and 280 nm. Eluting peaks were scanned between 200 nm and 300 nm with 1 nm intervals to determine absorbance maxima and minima.

By repeating the purification process using HPLC, a single compound was eluted at approximately 9.4 minutes with UV absorbance maximized at 290 and 325 nm, which revealed that it has a conjugated aromatic system. This compound (B22EES1-8-3) showed anti-inflammatory activities.

### Elucidation of the Molecular Structure

The structure of the resulting pure compound (B22EES1-8-3) was elucidated by using a Bruker 500 MHz DRX NMR spectrometer, operating at 500 MHz for ¹H and at 125.765 MHz for ¹³C NMR, using methanol-d as the solvent. Distortionless enhancement by polarization transfer (DEPT) experiments were performed using a transfer pulse of 135° to obtain positive signals for CH and CH₃, and negative signals for CH₂. HR-ESI-MS was performed on a micrOTOF II 411 ESI-TOF mass spectrometer (Bruker Daltonics). Data sets were acquired in negative electrospray (ESI) mode in a scan ranging from 100 to 1600 m/z at a sampling rate of 2 Hz. ESI parameters were as follows: capillary, 3.2 kV; nebulizer pressure, 4 bar; dry 415 gas flow, 8 L/min; and dry gas temperature, 200° C.

The ¹³C NMR spectra of the compound showed signals at δ 68.6 (t, C-1), 204.6 (s, C-2), 46.4 (t, C-3), 126.1 (s, C-4), 117.7 (d, C-5), 146.7 (s, C-6), 145.8 (s, C-7), 116.7 (d, C-8), 122.1 (d, C-9), 168.3 (s, C-1′), 115.3 (d, C-2′), 147.6 (d, C-3′), 128.9 (s, C-4′), 114.9 (d, C-5′), 148.2 (s, C-6′), 151.8 (s, C-7′), 112.6 (d, C-8′), 123.1 (d, C-9′), and 56.5 (q, MeO-7′). In addition, the compound showed a [M]− ion peak at m/z 357.0952 in its HR-ESI-MS, consistent with the molecular formula C19H17O7 (calc. 357.0974).

Determination of the Presence of B22EES1-8-3 in C. foetida and C. heracleifolia Using HPLC-UV and UPLC-TOF-MS

Herbs C. foetida and C. heracleifolia were extracted following the extraction procedure of C. racemosa as described above. The extracts of Herbs C. foetida and C. heracleifolia (CF22EES1-8 and CH22EES1-8) were injected into the HPLC equipped with a PDA detector following the chromatographic conditions that were used to isolate B22EES1-8-3. The chromatogram of individual sample was recorded. CF22EES1-8 and CH22EES1-8 were also injected separately into an Acquity UPLC system (Waters, USA) equipped with an Xterra MSC18 column (150*2.1 mmID, 3.5522 μm). Chromatographic separations were performed using a gradient elution from 25% acetonitrile (CH₃CN) to 90% CH₃CN at a flow rate of 200 μL/min. Eluted compounds were detected using a micrOTOF II ESI-TOF mass spectrometer (Bruker Daltonics). Data sets were acquired in negative electro spray (ESI) mode in a scan ranging from 100 to 1600 m/z at a sampling rate of 2 Hz. ESI parameters were as follows: capillary, 3.2 kV; nebulizer pressure, 4 bar; dry gas flow, 8 L/min; and dry gas temperature, 200° C.

By comparing their peaks with the standard of B22EES1-8-3, the presence of B22EES1-8-3 in the extract of C. foetida and C. heracleifolia was determined.

### Chemicals

Endotoxin (lipopolysachamide, LPS) from E. coli was purchased from Sigma and used as an inducer of TNF-α expression. Dexamethasone (Sigma) was used as a control drug to inhibit the LPS induction of TNF-α.

Cell Culture and Primary Blood Macrophage Isolation Human peripheral blood monocytic cells (PBMC) were isolated from the buffy coat of healthy donor blood supplied by Hong Kong Red Cross by Ficoll-Paque (Amersham Pharmacia Biotech, Piscataway, N.J.) density gradient centrifugation as described in our previous reports³⁶⁻³⁸. In brief, the buffy coat was spun at 3000 rotations per min (rpm) for 15 min to separate the blood cells and the plasma. The heat inactivated serum was filtered for future use.

The cell layer was diluted with phosphate buffered saline (PBS) in a ratio of 1:1. The diluted cells were overlaid on Ficoll-Paque slowly and centrifuged at 2300 rpm for 20 min for separation of mononuclear cells from erythrocytes. The mononuclear cell layer was removed and washed with RPMI 1640 medium until the supernatant was clear.

The cells were finally resuspended in RPMI 1640 medium supplemented with 5% autologous serum and cultured for 1 hr. The non-adherent cells were removed afterwards and the remaining adherent cells were further incubated for another 24 hr at 37° C. in 5% carbon dioxide (CO₂).

The adherent monocytic cells were detached and seeded onto tissue culture plates and incubated for another 7-14 days in order to differentiate the primary blood monocytic cells to primary blood macrophages (PBMac).

### Isolation of RNA and Reverse Transcription

Total RNA from primary blood macrophages with or without treatment of Cimicifuga racemosa fractions was extracted by TRIzol (Invitrogen). Reverse transcription (RT) of messenger RNA (mRNA) to complementary DNA (cDNA) was done by using the SuperScript II system (Invitrogen) as per the manufacturer's instruction.

### Polymerase Chain Reaction (PCR) and Real-Time RT-PCR

Semi-quantitative PCR assays of targeted genes were performed in a 25 μl reaction mixture containing 1.5 mM MgCl₂, 0.2 mM of each deoxynucleoside triphosphate, 0.25 μM of each primer, 2 units of Taq polymerase (Amersham Pharmacia Biotech, Piscataway, N.J.), and 1 μl of cDNA. PCR primer sets for TNF-α and glyceraldehyde-3-phosphate dehydrogenase (GAPDH) were as follows. TNF-α (upstream: 5′-GGCTCCAGGCGGTGCT TGTCC-3′ (SEQ ID NO:1); downstream: 5′-AGACGGCGATGCGGCTGATG-3′ (SEQ ID NO:2)), and GAPDH (upstream: 5′-ACCACAGTCCATGCCATCAC-3′ (SEQ ID NO:3); downstream: 5′-TCCACCACCCTGTTGCTGTA-3′ (SEQ ID NO:4). The thermal cycling condition for PCR was 94° C. for 30 s, 6° C. for 30 s, and 72° C. for 1 min. The cycling reactions were repeated for 24 more cycles.

Quantitative RT-PCR was performed according to the manufacturer's instructions by using Applied Biosystems TaqMan® Universal Master Mix. The TNF-α TaqMan® probes were purchased from the Applied Biosystems, and 18s RNA was used as an internal control. Samples were allowed to run in triplicates in each Quantitative RT-PCR assay.

### Enzyme-Linked ImmunoSorbent Assay (ELISA)

Culture supernatants of the LPS-treated PBMac, with or without B22EES1-8-3 pretreatment, were collected at different time intervals and stored at −70° C. The levels of the secreted TNF-α were measured by ELISA kits specific for the cytokine (R&D system, Minneapolis, Minn.).

### Preparation of Cellular Extracts

For the collection of whole cell lysate, PBMac were washed with cold PBS and incubated in cold lysis buffer (50 mM tris(hydroxymethyl)aminomethane-chloride (Tris-Cl) [pH7.4]; 150 mM sodium chloride (NaCl); 50 mM sodium fluoride (NaF); 10 mM β-glycerophosphate; 0.1 mM ethylenediaminetetraacetic acid (EDTA); 10% glycerol; 1% Triton X-100; 1 mM phenylmethanesulphonylfluoride (PMSF); 1 mM sodium orthovanadate; 2 μg/mL pepstatin A; 2 μg/mL aprotinin and 2 μg/mL leupeptin) for 20 min. The lysate was then centrifuged at 4° C. for 20 min. The supernatant was collected and stored at −70° C. until use.

To collect nuclear protein extracts, the treated cells were washed with PBS and resuspended in buffer A (10 mM 4-(2-hydroxyethyl)-1-piperazineethanesulfonic acid (HEPES) [pH7.9], 10 mM potassium chloride (KCl), 0.1 mM EDTA, 0.1 mM ethylene glycol tetraacetic acid (EGTA), 1 mM dithiothreitol (DTT), 0.5 mM phenylmethanesulphonylfluoride or phenylmethylsulphonyl fluoride (PMSF), 2 μg aprotinin, 1 mM sodium orthovanadate, 2 μg/mL pepstatin A, 2 μg/mL leupeptin and 50 mM NaF) for 15 min. After that, NP-40 at a final concentration of 0.625% was added and mixed vigorously for cell lysis.

The cell lysate was centrifuged and the supernatant containing cytoplasmic proteins was collected for storage at −70° C. The nuclear pellet was resuspended in buffer C (20 mM HEPES [pH 7.9], 0.4 M NaCl, 1 mM EDTA, 1 mM EGTA, 1 mM DTT and 1 mM PMSF) for 15 min on ice to complete lysis of the nuclear membrane. The nuclear lysate was then centrifuged, and the supernatant containing the nuclear protein was collected and stored at −70° C.

### Western Blot Analysis

Whole cell lysate (20 μg) or nuclear protein (2 μg) were separated by sodium dodecyl sulfate polyacrylamide gel electrophoresis (SDS-PAGE) and transferred to nitrocellulose membranes for probing overnight with the respective antibodies specific for the phosphorylated or total form of ERK1/2 and p38 MAPK (Cell Signaling Technology, Beverly, Mass.), NF-KB p65 protein and lamin B (Santa Cruz Biotechnology, Santa Cruz, Calif.). The membranes were incubated with the corresponding secondary antibodies conjugated with horseradish peroxidase (BD Transduction Lab, San Diego, Calif.). The signal was visualized by using enhanced chemiluminescence kit (Amersham Pharmacia Biotech). In order to quantify the results from the Western blots, the gels were scanned and the intensity of the bands was analyzed by a computer program Quantity One from BioRad.

### 5-LOX Inhibitor Screening Assay

The 5-LOX inhibition assay was performed using Cayman's Lipoxygenase Inhibitor Screening Assay Kit (Cayman Chemical Company, Ann Arbor, Mich.) according to the manufacturer's instructions. The 5-LOX enzyme and Linoleic acid (Substrate) were added together in the presence of DMSO (Control) and Cim A at a concentration ranging from 12.5 to 50 μg/ml. After incubation for 5 min, developing agents were added. The production of hydroperoxides was measured and quantified with the use of a microplate reader at wavelength of 490 nm. The 5-LOX enzymatic activity is calculated as nmol/min/ml.

### Leukotriene B₄ Production in Activated Human Blood Mononuclear Cells

Freshly isolated human blood mononuclear cells (PBMC) (1×10⁷) were pre-treated with 0.05% DMSO or Cim A (25 or 50 μg/ml) for 1 h, followed by addition of 5 μM Calcium ionophore A23187 (Sigma Aldrich, St. Louis, Mo.) for 30 min. After centrifugation for 1 min, the supernatants were collected. The level of leukotriene B₄ (LTB₄) was measured using LTB₄ parameter assay kit (R&D Systems, Minneapolis, Minn.).

The scope of the invention is not limited by the specific examples and suggested procedures and uses related herein since modifications can be made within such scope from the information provided by this specification to those skilled in the art.

A more complete understanding of the invention can be obtained by reference to the following specific examples of compounds, compositions, and methods of the invention. The following examples illustrate procedures for practicing the invention. These examples should not be construed as limiting. It will be apparent to those skilled in the art that the examples involve use of materials and reagents that are commercially available from known sources, e.g., chemical supply houses, so no details are given respecting them.

### Example 1

### Prediction of Cimiracemate A as a Lipoxygenase Inhibitor

The Similarity ensemble approach (SEA), a search tool provided by the Shoichet Laboratory in the Department of Pharmaceutical Chemistry at the University of California, San Francisco (UCSF), quantitatively groups and relates target protein pharmacology based on the set-wise chemical similarity among their ligands^{1,10}. The similarity score between ligand sets is expressed as expectation value (E-value), which can be used to complement scores of chemical similarity generated by BLAST^{1, 10-11}.

SEA search was performed using Cim A as the query compound, and E-values between Cim A and ligands of the target proteins were generated. The Tanimoto coefficient (Tc) of chemical similarity was also calculated. E-value <1×10⁻¹⁰ indicates significant similarity, whereas E-value >1.0 indicates insignificant similarity. Tc between 0 to 0.5 indicates insubstantial similarity, whereas Tc>0.5 indicates substantial similarity.

The SEA results suggest that Cim A has inhibitory effects against glucuronidase (beta), lipoxygenase (LOX), aldose reductase, cyclooxygenase, HIV integrase, adrenergic (beta1), and phospholipase A2. Specifically, E-values of Cim A as glucuronidase (beta) inhibitors and lipoxygenase inhibitors are 2.23×10⁻¹⁶ and 8.65×10⁻⁹, respectively (Table 1), suggesting significant similarity between Cim A and glucuronidase (beta) inhibitors as well as lipoxygenase inhibitors. Tc value of lipoxygenase inhibitors is 0.53—the highest Tc values of all protein inhibitors (Table 1), also suggesting Cim A as a lipoxygenase inhibitor.

TABLE 1
Prediction of Pharmacological Action of Cim A by SEA

| Rank | Pharmacological action | E-value | Max Tc |
|---|---|---|---|
| 1 | Glucuronidase (beta) Inhibitor | 2.23 × 10⁻¹⁶ | 0.37 |
| 2 | Lipoxygenase Inhibitor | 8.65 × 10⁻⁹ | 0.53 |
| 3 | Aldose Reductase Inhibitor | 4.77 × 10⁻⁴ | 0.38 |
| 4 | Cyclooxygenase Inhibitor | 1.87 × 10⁻³ | 0.50 |
| 5 | HIV Integrase Inhibitor | 9.45 × 10⁻³ | 0.44 |
| 6 | Adrenergic (beta1) Blocker | 9.00 × 10⁻² | 0.40 |
| 7 | Phospholipase A2 Inhibitor | 1.27 × 10⁻¹ | 0.41 |
| 8 | Tyrosine-Specific Protein Kinase | 1.81 | 0.37 |
| | Inhibitor | | |
| 9 | Dopamine Agonist | 3.18 | 0.33 |
| 10 | Phosphodiesterase Inhibitor | 8.89 | 0.35 |

### Example 2

### Determination of Inhibitory Effects of Cimiracemate A on 5-Lipoxygenase Activity

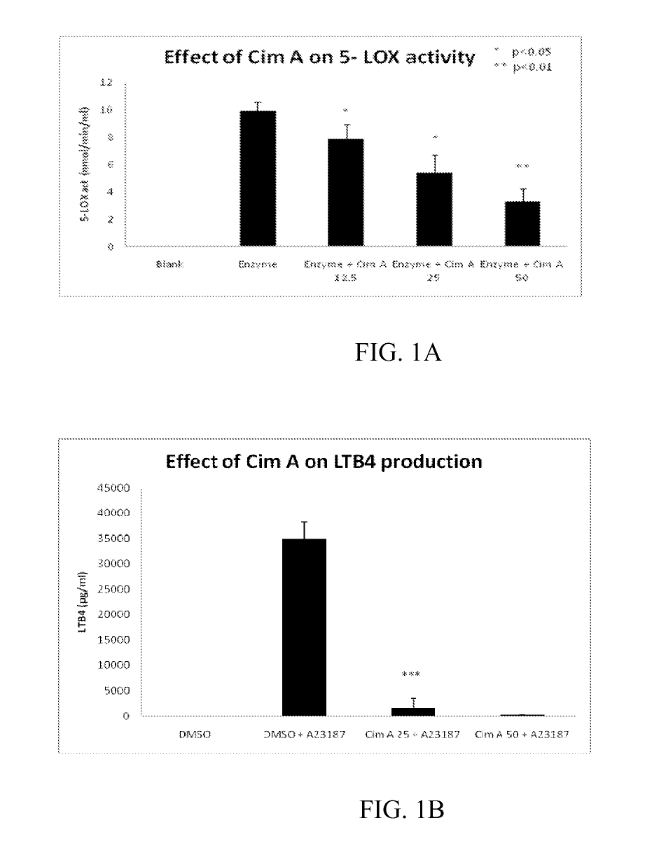

This Example demonstrates that Cim A potently inhibits 5-LOX activity. Briefly, 5-LOX inhibition assay was performed by using Cayman's lipoxygenase inhibitor screening assay kit (Cayman Chemical Company, Ann Arbor, Mich.) according to the manufacturer's instructions. The 5-LOX and linoleic acid (substrate) were added simultaneously into dimethyl sulfoxide (DMSO) (Control) or Cim A at a concentration of 12.5, 25, and 50 μg/ml, respectively. After incubation for 5 min, developing reagents for the 5-LOX inhibition assay were added. Hydroperoxide production was measured using a microplate reader at a wavelength of 490 nm. The 5-LOX enzymatic activity is calculated as nmol/min/ml. The results showed that Cim A potently suppressed 5-LOX activity in a dose-dependent manner (FIG. 1A).

### Example 3

### Determination of Inhibitory Effects of Cimiracemate A on LTB₄ Production

This Example investigates the effects of Cim A on LTB₄ production. Briefly, freshly isolated human blood mononuclear cells (1×10⁷) were pretreated with 0.05% DMSO or Cim A (25 or 50 μg/ml) for 1 hour, followed by the addition of calcium ionophore A23187 at a concentration of 5 (Sigma Aldrich, St. Louis, Mo.) for 30 min. After centrifugation for 1 min, the supernatant was collected. The level of leukotriene B₄ (LTB₄) was measured by using a LTB₄ assay kit (R&D Systems, Minneapolis, Minn.). The results, as shown in FIG. 1B, demonstrate that Cim A inhibits the production of LTB₄ in a dose-dependent manner.

### Example 4

### Extraction of Cim A from Cimicifuga Species

A light brown powder was obtained by repeated partitioning of the EtOAc fraction prepared from the rhizomes of Cimicifuga racemosa and sequential chromatography on silica gel and reversed-phase HPLC. The detailed procedures are summarized in FIG. 2.

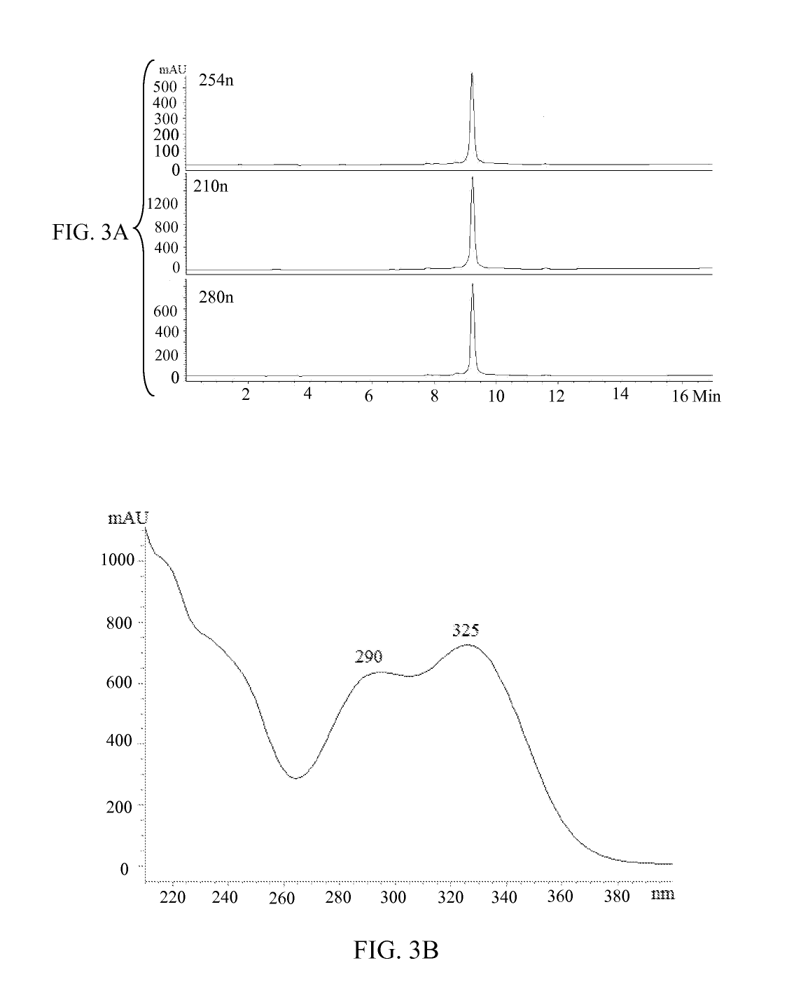

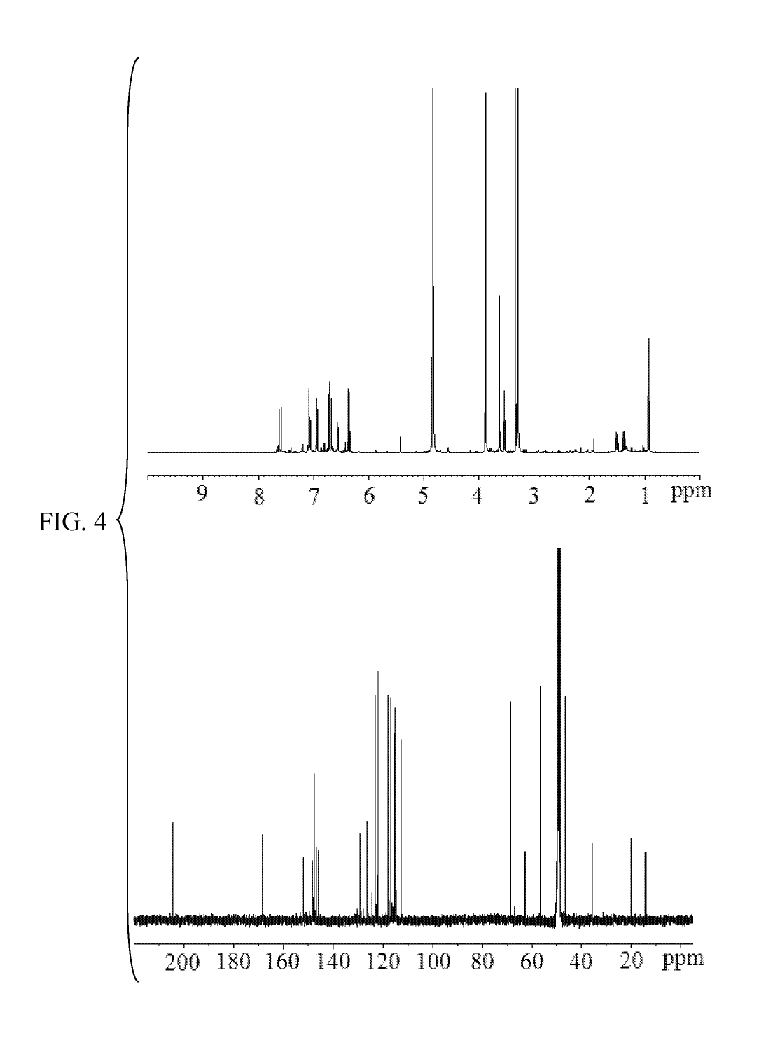

Using HPLC, the compound was eluted at approximate 9.4 min as a single compound with UV absorbance at wavelength 254, 210 and 280 nm (FIG. 3A). In FIG. 3B, the UV absorbance of the compound maximized at 290 and 325 nm, which revealed that it has a conjugated aromatic system. The compound showed a [M]⁻ ion peak at m/z 357.0952 in its HR-ESI-MS. Together with the ¹H and ¹³C spectra data (FIG. 4), it was elucidated as Cim A (B22EES1-8-3).

### Example 5

### Bio-Assays

The chemical compound in Cimicifuga racemosa responsible for the inhibition of LPS-induced expression of TNF-α was identified. LPS is well known to be a potent inducer of TNF-α and its effects cannot be easily suppressed without the use of cytotoxic agents.

Bacterial endotoxin (lipopolysaccharide, LPS) stimulation of TNF-α induction in primary macrophages was used as a model of inflammatory diseases, since the production of TNF-α is an indicator of a key immune response.

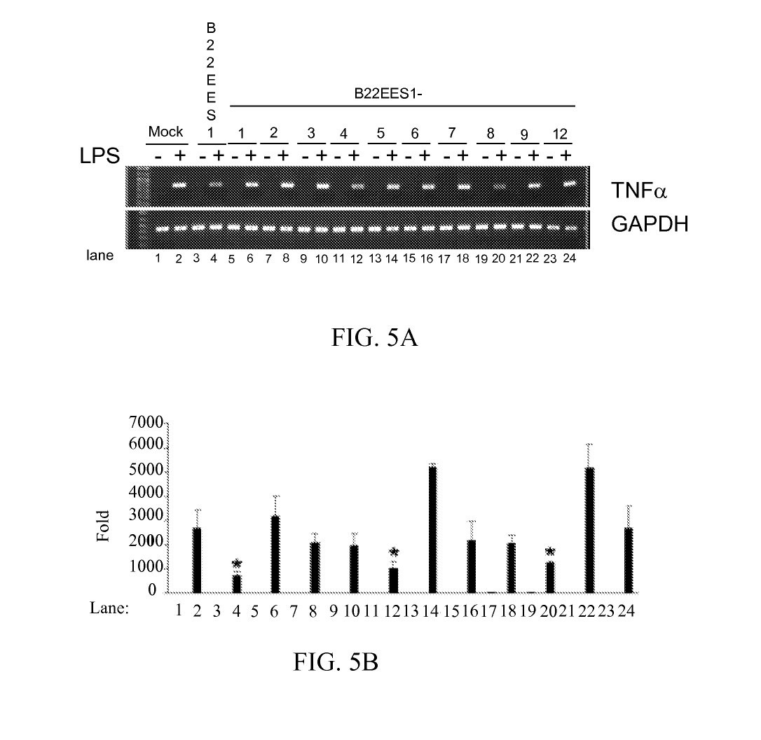

Individual extracts isolated from Cimicifuga racemosa were incubated with PBMac for 24 hr prior to the addition of LPS for another 3 hr. Total RNA of the treated samples was isolated and subjected to RT-PCR assays using specific human TNF-α primers. The results showed that the fraction B22EES1 inhibits LPS-induced TNF-α mRNA expression (FIG. 5A, lanes 2 and 4). Among the sub-fractions of B22EES1, only B22EES1-4 and B22EES1-8 retained the suppressive activities for TNF-α induction (FIG. 5A, lanes 12 and 20).

### Example 6

### Effects of Cim A on LPS-Induced Cytokine Production

After the identification of B22EES1-8 as being responsible for the inhibitory effects on TNF-α, the activities of B22EES1-8 sub-fractions as described above were separated and analyzed. A single molecule, namely Cim A (B22EES1-8-3), was found to be the active compound in the herbal extract responsible for the anti-inflammatory effects.

To confirm the activities of Cim A in suppressing TNF-α production, Cim A was incubated with PBMac for 24 hr prior the addition of LPS at concentrations of 1 ng/mL and 10 ng/mL for 24 hr. The culture supernatants were collected and measured by ELISA for the level of secreted TNF-α.

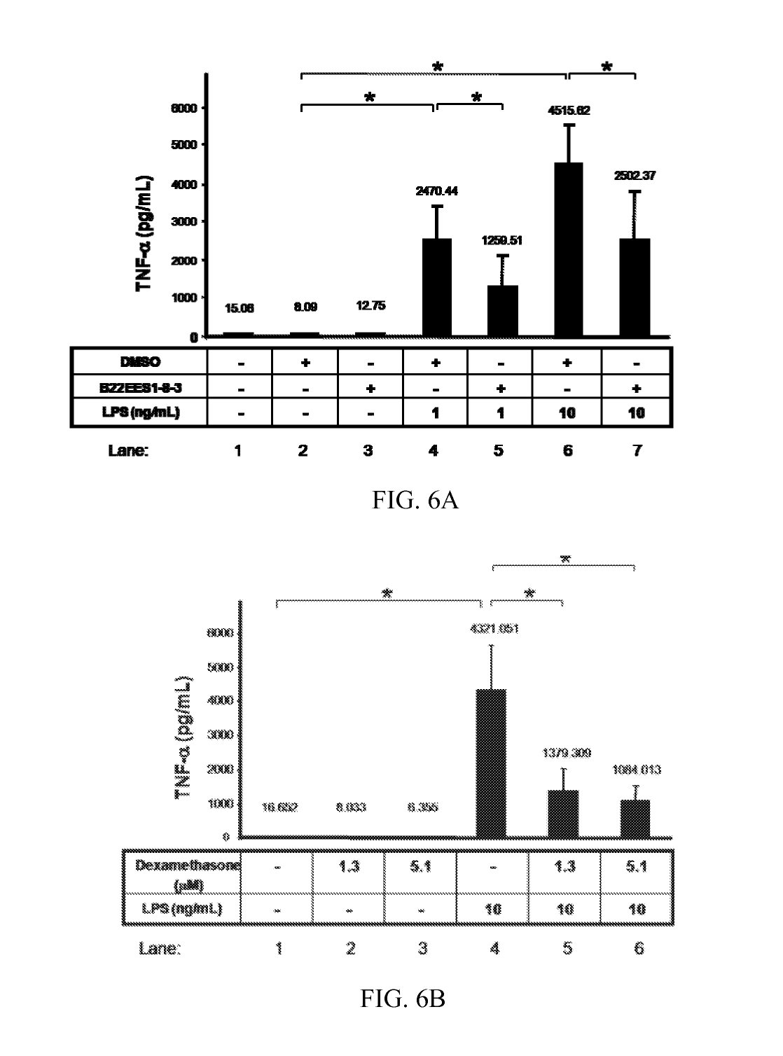

Cim A inhibited the LPS-induced TNF-α protein production by 47±19% and 58±30% at LPS concentrations of 1 ng/mL and 10 ng/mL, respectively (FIG. 6A, lanes 4 vs 5 and lanes 6 vs 7).

To further compare the efficiency of Cim A with existing drugs, dexamethasone, a potent immunosuppressive corticosteroid, was used as a prototype. PBMac were treated with dexamethasone for 24 hr prior to the addition of LPS at concentrations of 1 ng/mL and 10 ng/mL for 24 hr.

The results demonstrate that dexamethasone causes a significant inhibition of LPS-induced TNF-α production by 32±7.5% and 25±6.3% at concentrations of 1.3 and 5.1 μM, respectively (FIG. 6B).

### Example 7

### Molecular Mechanisms of Cytokine Downregulation by Cim A

The molecular pathways involved in Cim A inhibition of LPS-induced TNF-α production were elucidated. It is well documented that the activation of cytokine production in LPS-treated cells is initiated by the binding of LPS to its receptor³⁹. After binding to the receptor, a cascade of signaling kinases is activated. Among the activated kinases, MAP kinases play a crucial role in LPS-induced cytokine production. Previous studies illustrated that the induction of TNF-α by LPS and other pathogens requires the phosphorylation and activation of ERK1/2 and p38 MAPK^{36, 38, 40}.

In order to study the role of MAP kinases in Cim A inhibition of TNF-α production, PBMac were treated with Cim A for 24 hr and followed by the addition of LPS for 15 min. Protein samples were collected afterward and Western blots were performed.

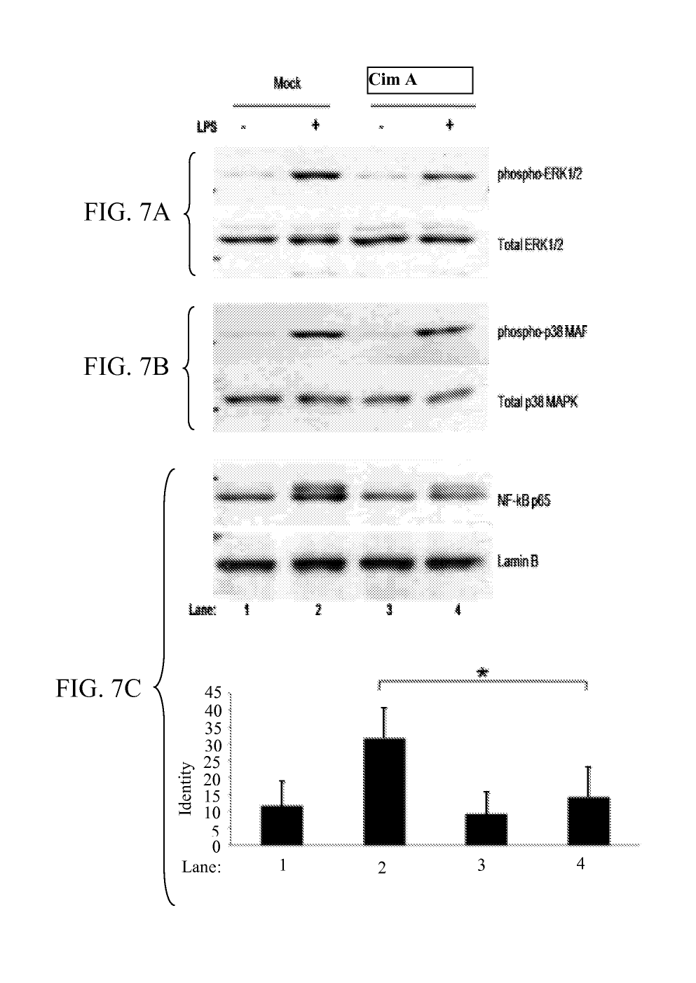

The results showed that LPS treatment results in phosphorylation of two different MAP kinases, namely ERK1/2 and p38 MAPK (FIG. 7. lane 2). With Cim A pretreatment, the phosphorylation of ERK1/2 (FIG. 7A, lanes 2 vs 4) but not p38 MAPK induced by LPS was suppressed (FIG. 7B, lanes 2 vs 4).

These results demonstrated that the anti-inflammatory activity of Cim A may be in part due to its inhibition of ERK1/2 phosphorylation.

Along the signaling pathways regulated by MAP kinases in response to LPS treatment, activation of the transcription factor NF-KB plays a critical role in the induction of proinflammatory cytokines including TNF-α⁴¹. The activation of NF-KB involves degradation of its specific inhibitor IKB and translocation of NF-KB sub-units from the cytoplasm to the nucleus. In accordance with the present invention, the addition of Cim A for 24 hr prior the addition of LPS reduced the translocation of NF-KB p65 subunit into the nucleus.

The results showed that the addition of Cim A to PBMac for 24 hr prior to the addition of LPS reduced the amount of p65NF-kB in the nuclear fraction (FIG. 7C, lanes 2 vs 4), indicating that the translocation of the p65NF-kB to the nucleus was inhibited by Cim A. In general, Cim A can inhibit LPS-induced kinase activities and their consequent activation of the nuclear transcription factor for TNF-α transcription. Thus, the compounds of the present invention can be used to regulate intracellular and/or extracellular activities that are downstream from NF-kB and/or ERK1/2 in the cascade of cellular events associated with inflammatory conditions.

### Example 8

### Determination of the Presence of Cim A in Cimicifuga foetida and Cimicifuga heracleifolia Using HPLC-UV

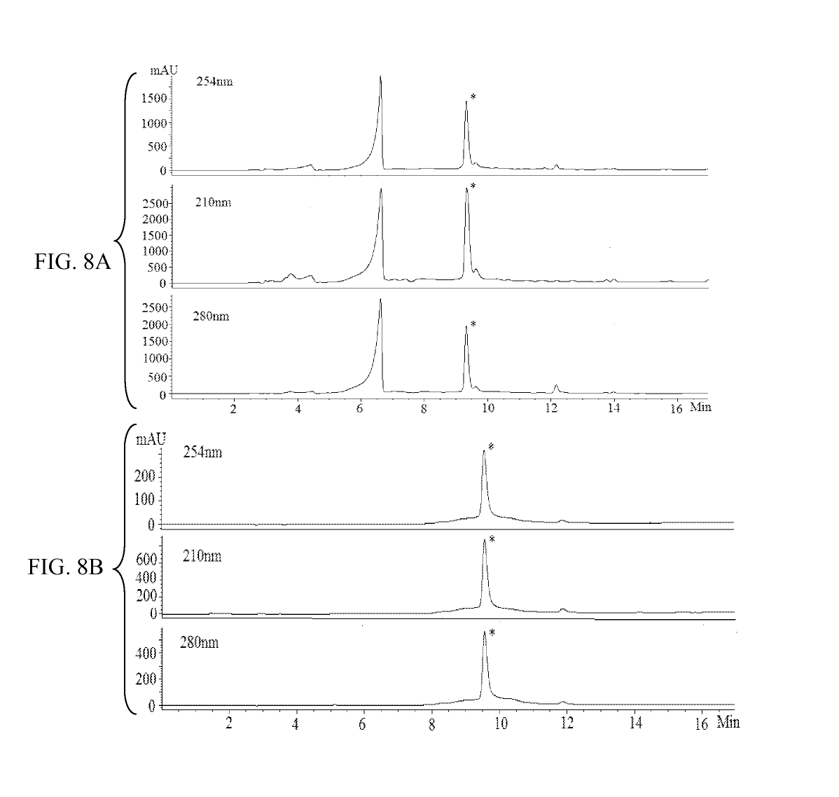

Under the same HPLC conditions, the retention time and the UV absorbance of Cim A were compared with the characteristic peak in the chromatograms of CF22EES1 and CH22EES1-8. In FIGS. 8A and B, both samples had a peak with retention time at approximate 9.4 min and their respective UV absorbance was same as that of Cim A (FIGS. 3A & B). The results revealed that herbs including C. foetida and C. heracleifolia contained Cim A.

### Example 9

### Determination of the Presence of Cim A in Cimicifuga foetida and Cimicifuga Heracleifolia Using UPLC-TOF-MS

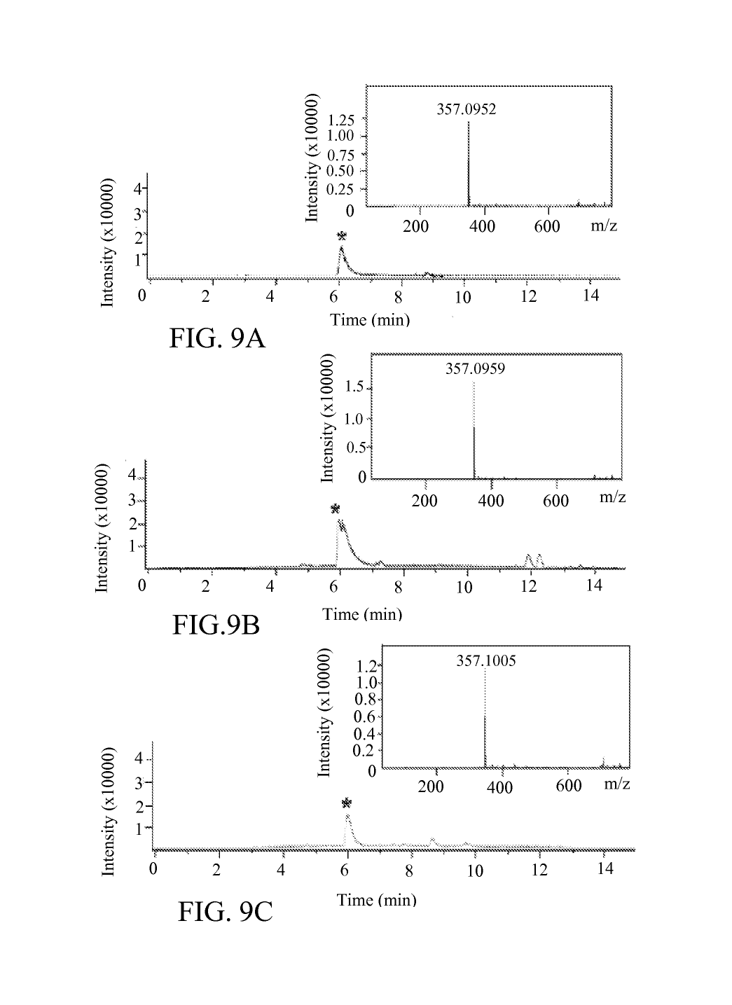

Under the same UPLC and ESI-MS conditions, the retention time and the mass-to-charge ratio of B8-3 (Cim A) were compared to the characteristic peak in the chromatograms and spectra of CF22EES1-8 and CH22EES1-8. In FIGS. 9B and C, both samples had a peak with retention time at approximate 6 min with an ion peak at m/z 357 that was the same as that of Cim A (FIG. 9A). The results revealed that herbs including C. foetida and C. heracleifolia contained B8-3 (Cim A).

### REFERENCES

1. Keiser, M. J. et al., “Relating protein pharmacology by ligand chemistry,” Nature Biotechnol. Feb. 7, 2007, pp. 197-206, Vol. 25, No. 2.
2. Samuelsson, B., “Leukotrienes: Mediators of Immediate Hypersensitivity Reactions and Inflammation,” Science, May 6, 1983, pp. 568-575, Vol. 220, No. 4597.
3. Weissmann, G., “Prostaglandins as Modulators Rather than Mediators of Inflammation,” J. Lipid Mediators, March-April 1993, pp. 275-286, Vol. 6, Nos. 1-3.
4. Jampilek, J.; Dolezal, M.; Opletalova, V.; Hartl, J., “5-Lipoxygenase, Leukotrienes Biosynthesis and Potential Antileukotrienic Agents,” Curr. Med. Chem., Jan. 15, 2006, pp. 117-129, Vol. 13, No. 2.
5. Ding, X.-Z.; Hennig, R.; Adrian, T. E., “Lipoxygenase and cyclooxygenase metabolism: new insights in treatment and chemoprevention of pancreatic cancer,” Mol. Cancer, Jan. 7, 2003, pp. 1-12, Vol. 2, No. 10.
6. Pommery, N.; Taverne, T.; Telliez, A.; Goossens, L.; Charlier, C.; Pommery, J.; Goossens, J.-F.; Houssin, R.; Durant, F.; He'nichart, J.-P., “New COX-2/5-LOX Inhibitors: Apoptosis-Inducing Agents Potentially Useful in Prostate Cancer Chemotherapy,” J. Med. Chem., Oct. 30, 2004, pp. 6195-6206, Vol. 47, No. 25.
7. Mehrabian, M.; Allayee, H., “5-Lipoxygenase and atherosclerosis,” Curr. Opin. Lipidol. October 2003, pp. 447-457, Vol. 14, No. 5.
8. Zhao, L.; Funk, C. D., “Lipoxygenase Pathways in Atherogenesis,” Trends Cardiovasc. Med., July 2004, pp. 191-195, Vol. 14, No. 5.
9. Dwyer, J. H.; Allayee, H.; Dwyer, K. M.; Fan, J.; Wu, H.; Mar, R.; Lusis, A. J.; Mehrabian, M., “Arachidonate 5-Lipoxygenase Promoter Genotype, Dietary Arachidonic Acid, and Atherosclerosis,” N. Engl. J. Med. Jan. 1, 2004, pp. 29-37, Vol. 350, No. 1.
10. Altschul, S. F.; Gish, W.; Miller, W.; Myers, E. W.; Lipman, D. J., “Basic local alignment search tool,” J. Mol. Biol., Oct. 5, 1990, pp. 403-410, Vol. 215, No. 3.
11. Hert, J.; Keiser, M. J.; Irwin, J. J.; Oprea, T. I.; Shoichet, B. K., “Quantifying the Relationships among Drug Classes,” J. Chem. Inf. Model, Mar. 13, 2008, pp. 755-765, Vol. 48, No. 4.
12. Yang C L, Chik S C, Li J C, Cheung B K, Lau A S. “Identification of the bioactive constituent and its mechanisms of action in mediating the anti-inflammatory effects of black cohosh and related Cimicifuga species on human primary blood macrophages,” J. Med. Chem., Nov. 12, 2009, pp. 6707-6715, Vol. 52, No. 21.
13. Gueli N, Verrusio W, Linguanti A, De Santis W, Canitano N, Ippoliti F, Marigliano V, Cacciafesta M., “Montelukast therapy and psychological distress in chronic obstructive pulmonary disease (COPD): A preliminary report,” Arch Gerontol Geriatr., May 15, 2010 PMID: 20478636
14. Interactive inhibitory effects of formoterol and montelukast on activated human neutrophils. Gravett C M, Theron A J, Steel H C, Tintinger G R, Cockeran R, Feldman C, Anderson R. Eur Respir J. 2010 Apr. 22. [Epub ahead of print]PMID: 20413544
15. Targeting leukotrienes for the treatment of COPD? Drakatos P, Lykouras D, Sampsonas F, Karkoulias K, Spiropoulos K. Inflamm Allergy Drug Targets. 2009 September; 8(4):297-306. PMID: 19754414
16. Enhanced fracture repair by leukotriene antagonism is characterized by increased chondrocyte proliferation and early bone formation: a novel role of the cysteinyl LT-1 receptor. Wixted J J, Fanning P J, Gaur T, O'Connell S L, Silva J, Mason-Savas A, Ayers D C, Stein G S, Lian J B. J Cell Physiol. 2009 October; 221(1):31-9. PMID: 19544365.
17. Cysteinyl leukotrienes enhance the degranulation of bone marrow-derived mast cells through the autocrine mechanism. Kaneko I, Suzuki K, Matsuo K, Kumagai H, Owada Y, Noguchi N, Hishinuma T, OnoM. Tohoku J Exp Med. 2009 March; 217(3):185-91. PMID: 19282653
18. 15-lipoxygenase metabolites play an important role in the development of a T-helper type 1 allergic inflammation induced by double-stranded RNA. Jeon S G, Moon H G, Kim. Y S, Choi J P, Shin T S, Hong S W, Tae Y M, Kim S H, Zhu Z, Gho Y S, Kim Y K. Clin Exp Allergy. 2009 June; 39(6):908-17. Epub 2009 Feb. 25. PMID: 19260872
19. Montelukast inhibits neutrophil pro-inflammatory activity by a cyclic AMP-dependent mechanism. Anderson R, Theron A J, Gravett C M, Steel H C, Tintinger G R, Feldman C. Br J Pharmacol. 2009 January; 156(1):105-15. Epub 2008 Dec. 6. PMID: 19068077
20. Leukotriene modifiers in the treatment of cardiovascular diseases. Riccioni G, Capra V, D'Orazio N, Bucciarelli T, Bazzano L A. J Leukoc Biol. 2008 December; 84(6):1374-8. Epub 2008 Sep. 15. Review. PMID: 18794213
21. ATP-dependent transport of leukotrienes B4 and C4 by the multidrug resistance protein ABCC4 (MRP4). Rius M, Hummel-Eiscnbeiss J, Keppler D. J Pharmacol Exp Ther. 2008 January; 324(1):86-94. Epub 2007 Oct. 24. PMID: 17959747
22. Prophylactic potential of montelukast against mild colitis induced by dextran sulphate sodium in rats. Holma R, Salmenper?P, Virtanen I, Vapaatalo H, Korpela R. J Physiol Pharmacol. 2007 September; 58(3):455-67. PMID: 17928642
23. Blockade of avidity and focal clustering of beta 2-integrin by cysteinyl leukotriene antagonism attenuates eosinophil adhesion. Meliton A Y, Munoz N M, Leff A R. J Allergy Clin Immunol. 2007 December; 120(6):1316-23. Epub 2007 Sep. 29. PMID: 17904626
24. What's new in paediatric sleep? Fauroux B. Paediatr Respir Rev. 2007 March: 8(1):85-9. Epub 2007 Mar. 21. Review. PMID: 17419982
25. The role of leukotrienes in the pathophysiology of inflammatory disorders: is there a case for revisiting leukotrienes as therapeutic targets? Sharma J N, Mohammed L A. Inflammopharmacology. 2006 March; 14(1-2):10-6. Review. PMID: 16835707
26. Montelukast protects against renal ischemia/reperfusion injury in rats. Sener G. et al., Pharmacol Res. 2006 July; 54(1):65-71. Epub 2006 Mar. 2. PMID: 16584888
27. Leukotriene receptor antagonists in children with cystic fibrosis lung disease: anti-inflammatory and clinical effects. Schmitt-Groh?S, Zielen S. Paediatr Drugs. 2005; 7(6):353-63.
28. Montelukast: new therapeutic option in patients with nasal polyps associated to respiratory allergic disease, Almeida Arvizu V et al., Rev Alerg Mex. 2005 July-August; 52(4):151-8. PMID: 16268183
29. Effect of montelukast on exhaled leukotrienes and quality of life in asthmatic patients. Biernacki W A, Kharitonov S A, Biernacka H M, Barnes P J. Chest. 2005 October; 128(4):1958 63. PMID: 16236841
30. Differential effect of zileuton, a 5-lipoxygenase inhibitor, against nociceptive paradigms in mice and rats. Singh V P, Patil C S, Kulkarni S K. Pharmacol Biochem Behav. 2005 July; 81(3):433-9. PMID: 15935457
31. Leukotriene modifier therapy for mild sleep-disordered breathing in children. Goldbart A D, Goldman J L, Veling M C, Gozal D. Am J Respir Crit Care Med. 2005 Aug. 1; 172(3):364-70. Epub 2005 May 5. PMID: 15879419
32. Exhaled breath condensate cysteinyl leukotrienes are increased in children with exercise-induced bronchoconstriction. Carraro S, Corradi M, Zanconato S, Alinovi R, Pasquale M F, Zacchello F, Baraldi E. J Allergy Clin Immunol. 2005 April; 115(4):764-70. PMID: 15805996
33. Antileukotriene drugs: clinical application, effectiveness and safety. Riccioni G, Bucciarelli T, Mancini B, Di Ilio C, D'Orazio N., Curr. Meda Chem. 2007: 14(18):1966-77.
34. Matsuyama et al., The target of 5-lipoxygenase is a novel strategy over human urological tumors than the target of cyclooxygenase-2, Drug Target Insights 2008: 3 137-151.
35. Burdette J E, Chen S N, Lu Z Z, Xu H, White B E, Fabricant D S, Liu J, Fong H S, Farnsworth N R, Constantinou A I, Van Breemen R V, Pezutto J M, Bolton J L. Black cohosh (Cimicifuga racemosa L.) protects against menadione-induced DNAdamage through scavenging of reactive oxygen species: bioassay-directed isolation and characterization of active principles. J Agric Food Chem 2002, 50: 7022-7028.
36. Li J C, Lee D C, Cheung B K, Lau A S. Mechanisms for HIV Tat upregulation of IL-10 and other cytokine expression: kinase signaling and PKR-mediated immune response. FEBS Lett. 2005; 579:3055-3062.
37. Lee D C, Cheung C Y, Law A H, Mok C K, Peiris M, Lau A S. p38 mitogen-activated protein kinase-dependent hyperinduction of tumor necrosis factor alpha expression in response to avian influenza virus H5N1. J Virol. 2005; 79:10147-10154.
38. Cheung B K, Lee D C, Li J C, Lau Y L, Lau A S A Role for Double-Stranded RNA-Activated Protein Kinase PKR in Mycobacterium-Induced Cytokine Expression. J Immunol. 2005; 175:7218-7225.
39. Lu Y C, Yeh W C, Ohashi P S. LPS/TLR4 signal transduction pathway. Cytokine. 2008; 42:145-151.
40. Kim S H, Kim J, Sharma R P. Inhibition of p38 and ERK MAP kinases blocks endotoxin-induced nitric oxide production and differentially modulates cytokine expression. Pharmacol Res. 2004; 49:433-439.
41. Blackwell T S, Christman J W. The role of nuclear factor-kappa B in cytokine gene regulation. Am J Respir Cell Mol Biol. 1997; 17:3-9.
42. Funk C D. Prostaglandins and leukotrienes: Advances in eicosanoid biology. Science 2001; 294: 1871-1875.
43. Wang Y, Mitchell J, Sharma M, Gabriel A, Moriyama K, Palmer P P. Leukotrienes mediate 5-hydroxytryptamine-induced plasma extravasation in the rat knee joint via CysLT-type receptors. Inflamm Res. 2004; 53: 66-71.44.
44. Tager A M, Dufour J H, Goodarzi K, Bercury S D, von Andrian U H, Luster A D. BLTR mediates leukotriene B(4)-induced chemotaxis and adhesion and plays a dominant role in eosinophil accumulation in a murine model of peritonitis. J Exp Med. 2000; 192: 439-46.
45. Jett M F, Marshall P, Fondacaro J D, Smith P L. Action of peptidoleukotrienes on ion transport in rabbit distal colon in vitro. J Pharmacol Exp Ther. 1991; 257: 698-705.
46. Goldenberg M M, Subers E M. The reactivity of rat isolated gastrointestinal tissues to leukotrienes. Eur J. Pharmacol. 1982; 78:463-6.
47. Helgadottir A, Manolescu A, Thorleifsson G, Gretarsdottir S, Jonsdottir H, Thorsteinsdottir U, Samani N J, Gudmundsson G, Grant S F, Thorgeirsson G, Sveinbjornsdottir S, Valdimarsson E. M, Matthiasson S E, Johannsson H, Gudmundsdottir O, Gurney M E, Sainz J, Thorhalisdottir M, Andresdottir M, Frigge M L, Topol E J, Kong A, Gudnason V, Hakonarson H, Guleher J R, Stefansson K. The gene encoding 5-lipoxygenase activating protein confers risk of myocardial infarction and stroke. Nat Genet. 2004; 36: 233-9.
48. Wallace J L, MacNaughton W K, Morris G P, Beck P L. Inhibition of leukotriene synthesis markedly accelerates healing in a rat model of inflammatory bowel disease. Gastroenterology 1989; 96: 29-36.
49. Fretland D J, Widomski D, Tsai B S, Zemaitis J M, Levin S, Djuric S W, Shone R L, Gaginella T S. Effect of the Leukotriene-B4 Receptor Antagonist Sc-41930 on Colonic Inflammation in Rat, Guinea-Pig and Rabbit. J Pharmacol Exp Ther. 1990; 255: 572-576.

We claim:

1. A method for reducing 5-lipoxygenase (5-LOX) activity, wherein said method comprises administering, to a subject in need of such treatment, an effective amount of an isolated compound or a prodrug thereof, wherein the compound has the following formula: 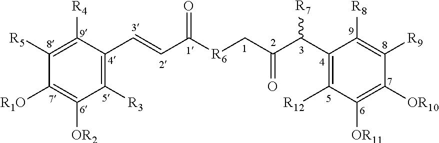
wherein
R₁ is alkyl;
R₂ is H or alkyl;
R₃, R₄, and R₅ are independently —H, acyl, halo, haloalkyl, amino, alkylamino, hydroxyl, alkyl, hydroxylalkyl, or —COOH;
R₆ is —O or —NH;
R₇ is —H, alkyl, alkoxy, hydroxylalkyl, hydroxyl, or halo;
R₈, R₉, and R₁₂ are independently —H, acyl, halo, amino alkylamino, hydroxyl, alkyl, hydroxylalkyl, or —COOH;
R₁₀ is H or alkyl; and
R₁₁ is H or alkyl;
wherein the method is used to treat a disease or condition selected from the group consisting of inflammation, allergy or allergenic reaction, cardiovascular disease, cerebrovascular disease, neoangiogenesis, respiratory or pulmonary disorder, skin disorder, asthma or asthma-related condition, arthritis, and cancer or tumor.

2. The method, according to claim 1, wherein the subject is a human.

3. The method, according to claim 1, wherein R₂ is H, R₃ is H, and R₄ is H.

4. The method, according to claim 3, wherein R₁ is a methyl group.

5. The method, according to claim 4, wherein the isolated compound is:

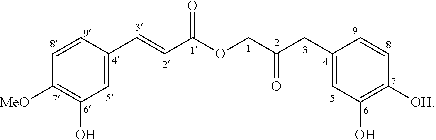 [SMILES: COc1ccc(/C=C/C(=O)OCC(=O)Cc2ccc(O)c(O)c2)cc1O]

6. The method, according to claim 1, wherein the compound is isolated from a Cimicifuga species.

7. The compound, according to claim 6, wherein the compound is isolated from a Cimicifuga selected from the group consisting of Cimicifuga racemosa, Cimicifuga foetida, and Cimicifuga heracleifolia.

8. The method, according to claim 1, used to reduce biosynthesis of LTA₄, LTB₄, LTC₄, LTD₄, and/or LTE₄.

9. The method, according to claim 8, used to reduce biosynthesis of LTB₄.

10. The method, according to claim 1, used to reduce cAMP or CGMP level.

11. The method, according to claim 1, used to reduce activity of aldose reductase, cyclooxygenase, HIV integrase, adrenergic (beta1), and/or phospholipase A2.

12. The method, according to claim 1, used to reduce biosynthesis of prostaglandins or thromboxane.

13. The method, according to claim 1, wherein the method is used to treat a disease selected from asthma, rhinitis, chronic obstructive pulmonary disease, cerebrovascular disease, or myocardial infarction.

14. The method, according to claim 1, wherein the method is used to treat bronchial asthma.

15. The method, according to claim 1, wherein the method is used to treat allergic rhinitis.

16. The method, according to claim 1, wherein the method is used to treat a cerebrovascular and/or cardiovascular disease selected from the group consisting of myocardial infarction, acute myocardial infarction, stroke, atherosclerosis, thrombosis, coronary angioplasty, angina, myocardial ischemia, hypertension, platelet aggregation, aortic aneurysms, vascular inflammation, intimal hyperplasia, or hyperlipidemia-dependent aortic aneurysm.

17. The method, according to claim 1, wherein the method is used to treat a respiratory or pulmonary condition selected from the group consisting of cystic fibrosis lung diseases, sleep-disorder breathing, obstructive sleep apnea (OSA), and chronic obstructive pulmonary disease (COPD).

18. The method, according to claim 1, wherein the method is used to treat tumor or cancer.

19. The method, according to claim 18, wherein the cancer or tumor is selected from the group consisting of urological tumor, renal cell carcinoma, bladder tumor, prostate cancer, and pancreatic cancer.

20. The method, according to claim 1, used to inhibit neoangiogenesis.


SEQUENCE LISTING (4 sequences)

SEQ ID NO: 1 - DNA, 21 bp, Artificial Sequence
  PCR Primer set for TNF-alpha (upstream)
ggctccaggcggtgcttgtcc

SEQ ID NO: 2 - DNA, 20 bp, Artificial Sequence
  PCR Primer set for TNF-alpha (downstream)
agacggcgatgcggctgatg

SEQ ID NO: 3 - DNA, 20 bp, Artificial Sequence
  PCR Primer set for GAPDH (upstream)
accacagtccatgccatcac

SEQ ID NO: 4 - DNA, 20 bp, Artificial Sequence
  PCR Primer set for GAPDH (downstream)
tccaccaccctgttgctgta